(19) United States
(12) Patent Application Publication
(10) Pub. No.: US 2021/0298816 A1
(43) Pub. Date: Sep. 30, 2021

(54) ELECTROSURGICAL SYSTEM, ELECTROSURGICAL INSTRUMENT AND ELECTROSURGICAL GENERATOR

(71) Applicant: OLYMPUS WINTER & IBE GMBH, Hamburg (DE)

(72) Inventors: Fabian JANICH, Potsdam (DE); Anne KWIK, Berlin (DE); Frank BREITSPRECHER, Berlin (DE); Jens KRÜGER, Eichwalde (DE); Karsten LORENZ, Teltow (DE); Michael KADEL, Berlin (DE)

(73) Assignee: OLYMPUS WINTER & IBE GMBH, Hamburg (DE)

(21) Appl. No.: 17/217,554

(22) Filed: Mar. 30, 2021

(30) Foreign Application Priority Data
Mar. 30, 2020 (DE) 102020108798.5
Mar. 31, 2020 (DE) 102020108970.8
Oct. 30, 2020 (DE) 102020128695.3

(51) Int. Cl. A61B 18/12; G16H 20/40; G16H 40/63

(52) U.S. Cl. CPC A61B 18/1206; A61B 2017/00119; G16H 40/63; G16H 20/40

(57) ABSTRACT

An electrosurgical system with an electrosurgical instrument and an electrosurgical generator to which the electrosurgical instrument is connected. The electrosurgical generator has a processor and at least one generator data memory as well as optical and our acoustic signaling and/or display means. The electrosurgical instrument has at least one instrument data memory in which a data set with structured data is stored, wherein at least some of this data represent signaling instructions. The generator data memory contains an operating program for controlling the operation of the electrosurgical generator in conjunction with the electrosurgical instrument and, by means of the operating program, the processor is configured to control during the operation of the electrosurgical generator the signals or notifications to be output by the signaling and/or display means in dependence on the data representing signaling instructions, which is stored in the instrument data memory of the electrosurgical instrument.

The invention relates to an electrosurgical system with an electrosurgical generator that is configured to supply high-frequency alternating current to an electrosurgical instrument. The invention also relates to a method of operating an electrosurgical system as well as an electrosurgical instrument and an electrosurgical generator.

An electrosurgical system generally comprises an electrosurgical generator for generating the high-frequency alternating current. As a general rule, the electrosurgical generator has two or more outputs where an electrosurgical instrument can be connected, and a high-frequency AC voltage is provided between these outputs during operation. In addition, an electrosurgical generator generally comprises a high-voltage power supply that generates direct current during operation, and a high-frequency part that is connected to the high-voltage power supply and generates a high-frequency alternating current from the direct current during operation.

Typically, different electrosurgical instruments can be connected to the electrosurgical generator for different tasks. On the other hand, an electrosurgical instrument may, under certain circumstances, also be able to be operated with different electrosurgical generators.

Electrosurgery can be used for cutting, coagulating (obliterating) and/or vaporizing biological tissue, i. e. body tissue. High-frequency alternating currents with a frequency between 0.2 MHz and 3 MHz are typically used in electrosurgery. Electrosurgical instruments are typically handheld instruments that a surgeon can use to coagulate, ablate and/or cut body tissue.

To this end, the electrosurgical instruments are supplied with high-frequency electrical energy, by means of which tissue can be coagulated or cut in a targeted manner. The high-frequency electrical energy is supplied by an electrosurgical generator and applied to the body tissue by means of the electrosurgical instrument that is appropriate in the respective case. Depending on what the electrosurgical instrument is used for, specific current and voltage curves are required; these current and voltage curves are made available to the physician for selection at the electrosurgical generator in the form of operating modes (also referred to as modes). These operating modes can be permanently stored in the electrosurgical generator.

A simple programmable supply device for an electrosurgical instrument is known from WO 2011/032729 A1. The simple programmability results from defined possible states of a state machine, which, however, also limit the flexibility of the supply device.

It is the object of the invention to create an electrosurgical system that offers improved options for delivering information to a user.

To this end, the invention proposes an electrosurgical system, an electrosurgical generator and an electrosurgical instrument which, on the one hand, can each individually and in combination with each other be flexibly combined, and which, on the other hand, allow for operating modes that are tailored to a particular application and/or to a particular electro-surgical instrument. Furthermore, a method of operating the electrosurgical system and the electrosurgical generator are proposed.

An electrosurgical system according to the invention comprises an electrosurgical instrument and an electrosurgical generator to which the electrosurgical instrument is connected during operation.

The electrosurgical generator:

The electrosurgical generator has a processor and at least one generator data memory that the processor can access and in which an operating program for controlling the operation of the electrosurgical generator in combination with the electrosurgical instrument is stored. Furthermore, the electrosurgical generator has outputs for connecting an electrosurgical instrument. An electrosurgical instrument connected in operation to the electrosurgical generator can be supplied with electrical energy, in particular with high-frequency AC voltage or high-frequency alternating current, via the outputs. The electrosurgical generator also has a generator data interface for data communication with an electrosurgical instrument, in particular a data connection.

The electrosurgical instrument:

The electrosurgical instrument has an instrument data memory in which at least one data set is stored; said data set containing structured data that is implemented as a plurality of parameterized references referring to the control commands stored in the generator data memory of the electrosurgical generator. Furthermore, the data set may comprise the operating parameter values. The electrosurgical instrument also has an instrument data interface for data communication with the electrosurgical generator. During operation, the electrosurgical generator can read structured data from the instrument data memory via the instrument data interface. In addition, the instrument data interface makes it possible to store data sets with structured data that is implemented as a plurality of parameterized references, in the instrument data memory.

The operating program:

In addition to triggering control interventions at the electrosurgical generator, the operating program is configured to trigger memory access to the structured data in the instrument data memory and to execute control commands stored in the generator data memory, which the parameterized references represented by the structured data refer to.

Procedure during the operation of the electrosurgical generator:

During the operation of the electrosurgical generator, the processor executes an operating program stored in the generator data memory and controls the electrosurgical generator in accordance with the operating program. Under the control of the operating program, the processor triggers in particular control interventions at the electrosurgical generator.

In this context, control interventions comprise for example the control of the delivery of energy to an electrosurgical instrument that is, during operation, connected to the electrosurgical generator. However, control interventions can also cause signals to be emitted or information to be displayed.

The control interventions can also be directly controlled by the operating program or depend on the parameterized references contained in the structured data stored in the instrument data memory. Values for parameters specifying control interventions can also be contained in the structured data in the instrument data memory.

During the operation of the electrosurgical generator, the parameterized references stored in the structured data are called up by the operating program and those of the control commands stored in the generator data memory that are referred to by the parameterized references represented by the structured data will be executed. If the structured data contains operating parameter values, these operating parameter values specified in the structured data will be observed in the execution of the control commands.

To this end, the electrosurgical generator accesses the structured data during operation under the control of the processor and the associated operating program. This access can be direct access to the instrument data memory. Alternatively, the operating program can also be configured to cause a transfer of the structured data from the instrument data memory into the generator data memory. This transfer can take place when the electrosurgical generator is started up or while the electrosurgical instrument is being connected. If the structured data is imported into the generator data memory when the electrosurgical generator is started or when an electrosurgical instrument is connected, the processor will access the structured data that is available in the generator data memory in this case.

The operating method for the electrosurgical system:

According to the invention, a corresponding method of operating an electrosurgical system with an electrosurgical instrument is proposed as well. The electrosurgical system has an electrosurgical generator to which the electrosurgical instrument is connected. The electrosurgical [ . . . ] has an instrument data memory. The electrosurgical generator has a processor and at least one generator data memory. In order to control the electrosurgical generator, the processor executes an operating program stored in the generator data memory.

According to the invention, a data set containing structured data is generated or provided, wherein the structured data is implemented as a plurality of parameterized references referring to operating specifications stored in the generator data memory of the electrosurgical generator. The operating specifications are in particular control commands that are potentially to be applied. In addition to the parameterized references, the structured data may also comprise operating parameter values. However, operating parameter values may also be part of the operating specifications stored in the generator data memory. In this case, the parameterized references may not only refer to control commands, but also to operating parameter values.

The data set with structured data is or will be stored in the instrument data memory of the electrosurgical instrument.

When the electrosurgical generator is started up or during its operation, the data set with structured data stored in the instrument data memory is read by the electrosurgical generator and the parameterized references stored in the structured data are called up under the control of the operating program for the execution of the control commands stored in the electrosurgical generator, wherein the operating parameters that might also be included in the structured data are observed, so that the electrosurgical generator is controlled in dependence on the operating program, the operating specifications (e.g. control commands) stored in the generator data memory and the parameterized references to individual ones of the control commands stored in the generator data memory which are contained in the structured data.

During the execution of the operating program, the operating program and the structured data preferably interact in such a way that the execution of the operating program and the call-up of the structured data by the operating program define the operation of the electrosurgical generator in an operating mode.

The data set with structured data can preferably be generated manually or with the help of a software.

Preferably, after being read by the electrosurgical generator, the data set with structured data stored in the instrument data memory of the electrosurgical instrument is stored in the generator data memory of said electrosurgical generator. Thus, the processor only needs to access the generator data memory in order to call up the operating program as well as the structured data and the operating specifications. At the same time, the advantages of a data set with structured data stored in the instrument data memory are preserved.

The operating method may also comprise steps in the context of which structured data stored on the electrosurgical instrument is changed while it is being read by the electrosurgical generator or after it has been stored in the generator data memory of the electrosurgical generator. Such changes can, for example, include the addition of line numbers and relative addresses.

The structured data may comprise signaling instructions that are converted into configuration and/or operating status-dependent acoustic and/or visual notifications to a user during the operation of the electrosurgical generator.

The structured data may comprise definitions for activation sounds.

The structured data may comprise conditions and characteristics for a specific notification or error message.

Preferably, the structured data comprises data representing control characters for text notifications, wherein the control characters define the display of text notifications on various displays and wherein text notifications are generated during the operation of the electrosurgical generator in dependence on the control characters and a display for text present at the electrosurgical generator.

The electrosurgical system:

According to the invention, an electrosurgical system comprising an electrosurgical instrument with at least one instrument data memory and an electrosurgical generator, to which the electrosurgical instrument is connected, is proposed as well. The electrosurgical generator has a processor and at least one generator data memory which the processor can access during operation. An operating program that the processor can execute to control the electrosurgical generator is stored in the generator data memory. In addition, operating specifications that define at least a plurality of control commands are stored in the generator data memory. In the context of this description, these operating specifications that define control commands are also referred to as control commands.

A data set with structured data is stored in the instrument data memory of the electrosurgical instrument. The structured data is implemented as a plurality of parameterized references that refer to the operating specifications that represent control commands and are stored in the generator data memory of the electrosurgical generator. In addition, the structured data may comprise operating parameters.

The electrosurgical generator is configured to read the data set of structured data stored in the instrument data memory of the electrosurgical instrument when the electrosurgical generator is started up or during its operation, and to call up the parameterized references stored in the structured data during operation using the operating program, in order to execute those of the control commands stored as part of the operating specifications in the electrosurgical generator that are referred to by a corresponding parameterized reference. For this, the operating parameters specified in the structured data can be taken into account. This means that, during operation, the electrosurgical generator is controlled in dependence on the operating program, the operating specifications stored in the generator data memory (control commands, in particular) and the parameterized references to individual ones of the operating specifications stored in the generator data memory which are contained in the structured data.

The electrosurgical generator may have optical and/or acoustic signaling and/or display means, in order to provide optical and/or acoustic notifications to a user during the operation of the electrosurgical generator. If the electrosurgical generator has optical and/or acoustic signaling and/or display means, the processor of the electrosurgical generator is, in combination with the operating program, configured to control signals or notifications to be output via the signaling and/or display means based on the structured data that is stored in the instrument data memory and represents signaling instructions. The signaling instructions represented by structured data stored in the instrument data memory define which signals and/or information are to be output by the electrosurgical generator at which the electrosurgical instrument is operated, and how and under what conditions these signals and/or information are to be output.

As part of the signaling means, the electrosurgical generator preferably has a sound generator and a loudspeaker connected to it for outputting acoustic signals. The sound generator is operatively connected to the processor and configured to generate acoustic signals and to output them via the loudspeaker under the control of the processor in dependence on data in the instrument data memory which represents signaling instructions.

The electrosurgical generator has preferably also a display as a signaling means for displaying text and/or symbols. The display is connected to the processor and configured to output text notifications under the control of the processor in dependence on data in the instrument data memory which represents signaling instructions.

Under the control of the operating program, the processor is, in particular, configured to read the data representing the signaling instructions from the instrument data memory and to process them taking into account the configuration and/or status information in such a way that the output of signals or information to a user is made in dependence on the status and/or the configuration in the manner defined in the electrosurgical instrument. Configuration information can for example concern other electrosurgical instruments connected to an electrosurgical generator. Status information can be information on an operating status of the electrosurgical generator obtained by sensors or status information generated by the processor during operation.

Signaling instructions can be read from the instrument data memory during the connection of the electrosurgical instrument to the electrosurgical generator or during the operation of the electrosurgical instrument.

The status- and/or configuration dependent output of signals or information to a user can include the presentation of texts on a display or the output of acoustic signals. This makes it possible to output warning and/or status information, e.g. concerning the operating mode, error messages, operating states etc., to a user.

The control commands and, if applicable, the operating parameter values referred to by the parameterized references are operating specifications that are stored in the generator data memory. Independently of the connection of an electrosurgical instrument, the generator data memory contains a theoretical arbitrary number of operating specifications that can be called up by the operating program and influence the operation of the electrosurgical generator, but which, by themselves, do not define any fixed operating sequences. The operating specifications can, on the one hand, be control commands, control expressions and/or conditional expression etc., but can, on the other hand, also be operating parameters such as values for the output voltage, the current amount, time settings, impedance values, desired values etc. The instrument data memory can, in particular, also contain audio files that define acoustic signals, which can be output during the operation of the electrosurgical generator under the control of the processor in conjunction with the operating program and in dependence on the data in the data storage of the electrosurgical instrument which defines the signaling instructions.

The operating specifications in the instrument data memory can be regarded as a library of any plurality of operating specifications (such as audio files for acoustic signals that may have to be output or control information for a text display or a frequency generator) which the operating program can access. However, these operating specifications are not accessed directly, so that the operating specifications and the operating program as such do not determine the mode of operation of the electrosurgical generator.

Instead, the processor, that is controlled by the operating program, accesses the structured data in the instrument data memory which represents the signaling instructions, during the connection of an electrosurgical instrument and/or during the operation, so that the output of signals or information to a user is performed in a manner that is defined in the electrosurgical instrument.

Preferably, audio files that the sound generator can—under the control of the processor in dependence on structured data in the instrument data memory which represents signaling instructions—convert into acoustic signals are stored in the generator data memory.

Preferably, a data structure in which arbitrary data sets with structured data are stored, is also provided. The structured data contains references to potentially applicable operating specifications such as control commands or operating parameter values. During the operation of the electrosurgical generator, the parameterized references defined by the structured data are called up by the operating program that is being executed at that time and translated into operating specifications that are specifically applicable and that the operating program is then able to apply. The structured data can, in particular, also be stored in the instrument data memory and contain data that represents the signaling instructions.

Thus, the specific mode of operation of the electrosurgical generator depends preferably on three different types of stored data, namely
on the data in the generator data memory which defines the operating program,
on the potentially applicable operating specifications in the generator data memory which defines for example control commands or operating parameter values, and
on the data sets with structured data stored in the data structure which, triggered by the operating program during its execution, cause the specifically applicable operating specifications to be called up from the potentially applicable operating specifications stored in the instrument data memory.

Some of the structured data stored in the data structure represents preferably signaling instructions that the processor of the electrosurgical generator converts during the operation of the electrosurgical generator into acoustic or optical signals and/or notifications for a user. The conversion of the signaling instructions into acoustic and/or optical signals and/or notifications for a user can be performed in a status- and/or configuration-dependent manner that is defined in the electrosurgical instrument, since the data representing the signal instructions can be stored in a data structure on the electrosurgical instrument.

The data defining the operating program and the data defining the operating specifications in the instrument data memory as well as the data structure with structured data can be changed and defined independently, as long as the data formats are compatible. Thus, the mode of operation of the electrosurgical generator in a respective operating mode can be changed through changes to the operating program stored in the instrument data memory, or through changes to the operating specifications stored in the instrument data memory, or through changes to the structured data in the data structure, or also through a combination of these changes.

The electrosurgical instrument contains the non-volatile instrument data memory (e.g. an EEPROM or similar), but no processor. The instrument data memory contains a data set with structured data. The structured data of this data set is compatible with the data structure in the generator data memory of the electrosurgical generator and may form the data structure of the electrosurgical generator or a part of the data structure of the electrosurgical generator.

Thus, the data structure can be available in the generator data memory, which is a physical component of the electrosurgical generator, or it is formed by the content of the instrument data memory or by a combination of the data in the generator data memory of the electrosurgical generator and the data in the instrument data memory of the electrosurgical instrument.

By means of the operating program in the generator data memory, the processor of the electrosurgical generator is configured to read the instrument data memory of the electrosurgical instrument.

By means of the operating program in the generator data memory, the processor of the electrosurgical generator may be configured to read the instrument data memory of the electrosurgical instrument after the electrosurgical instrument has been connected to the electrosurgical generator and before the electrosurgical generator is operated in an operating mode. By means of the operating program in the generator data memory, the processor of the electrosurgical generator may be configured to transfer the data set or the data sets or the structured data contained in the data set or the data sets into a data structure that is stored in the generator data memory of the electrosurgical generator.

By means of the operating program in the generator data memory, the processor of the electrosurgical generator may alternatively be configured to read the instrument data memory during the execution of the operating program—i.e. while the electrosurgical generator is being operated in an operating mode. Thus, no computer program or algorithm is stored in the instrument data memory, and the structured data is neither readable as a computer program nor as an algorithm associated with a program, but instead constitutes references to the operating specifications stored in the generator data memory.

The structure of the structured data makes it possible to assign line numbers to the parameterized references. With the help of the line numbers, the references associated with them can be called up in a targeted manner by means of the operating program. The references, in turn, unambiguously refer to specific operating specifications in the generator data memory or, alternatively, also to other references in the structured data.

Instead of the provision of line numbers that are explicitly stored in the data set, it may be provided that the line numbers are only generated when a data set is read, namely based on the structure of the structured data in a data set which represents the references. This is possible if the references are, for example, stored in a designated order in a data set.

The structured data is preferably available in a memory-efficient binary format that can, for example, be represented in the form of hexadecimal numbers.

The electrosurgical system has preferably a programming interface or several programming interfaces, by means of which the data structure can be programmed and transferred to an electrosurgical instrument. It may also be provided that the operating program and/or the operating specifications, in particular the control commands, in the generator data memory can be changed via a programming interface, for example by way of a software update of the electrosurgical generator via USB.

What is preferred is a method of operating an electrosurgical generator where the data representing the signaling instructions is read from an instrument data memory of an electrosurgical instrument connected to the electrosurgical generator and is, during the operation of the electrosurgical generator, converted into configuration- and/or operation-dependent acoustic and/or optical notifications for a user.

Preferably, the data representing signaling instructions for text notifications contains control characters that define the display of text notifications on various displays, so that text notifications are generated during the operation of the electrosurgical generator in dependence on the control characters and a display for text present at the electrosurgical generator.

Preferably, the data representing the signaling instructions is read from an instrument data memory of an electrosurgical instrument connected to the electrosurgical generator during the operation of the electrosurgical generator. Alternatively, the data representing the signaling instructions can also be read from the instrument data memory of the electrosurgical instrument while an electrosurgical instrument is being connected to the electrosurgical generator.

Preferably, operating specifications and a data structure are provided in memory areas of the generator data memory on the electrosurgical generator which are independent of each other, wherein the data structure contains references to the operating specifications.

Preferably, the processor of the electrosurgical generator indirectly accesses individual operating specifications during the execution of an operating program by accessing first of all parameterized references in the data structure and subsequently calling up the operating specification or the operating specifications referred to by the respective reference.

The data structure is preferably read from the instrument data memory of an electrosurgical instrument; preferably after an electrosurgical instrument has been connected and before it is used. The data sets with structured data contained in the data structure on the electrosurgical instrument can be transferred into a data structure that is stored in the generator data memory of the electrosurgical generator.

Another aspect of the invention is an electrosurgical generator for an electrosurgical system of the aforementioned type. The electrosurgical generator has connections for connecting an electrosurgical instrument as well as a processor and at least one generator data memory and optical and/or acoustic signaling and/or display means for sending optical and/or acoustic notifications to a user during the operation of the electrosurgical generator. The generator data memory contains an operating program for the processor for controlling the operation of the electrosurgical generator in conjunction with an electrosurgical instrument connected to the electrosurgical generator during operation. By means of the operating program, the processor of the electrosurgical generator is configured to control during the operation of the electrosurgical generator the signals or notifications to be output by the signaling and/or display means in dependence on structured data representing signaling instructions, which is stored in an instrument data memory of a connected electrosurgical instrument.

As signaling means, the electrosurgical generator preferably has a sound generator and a loudspeaker connected to it for outputting acoustic signals. The sound generator is connected to the processor of the electrosurgical generator and configured to generate acoustic signals and to output them via the loudspeaker under the control of the processor of the electrosurgical generator in dependence on data in the instrument data memory of the electrosurgical instrument which represents signaling instructions.

The electrosurgical generator has preferably also a display as a signaling means for displaying text and/or symbols. The display is connected to the processor of the electrosurgical generator and configured to output text notifications under the control of the processor of the electrosurgical generator in dependence on data in the data memory of the electrosurgical instrument which represents signaling instructions.

Such an electrosurgical generator can easily be used with a variety of different electrosurgical instruments, wherein the adaptation to the respective electrosurgical instrument can be made solely by means of corresponding entries in the data structure which represent references, without the operating specifications in the generator data memory of the electrosurgical generator having to be changed.

Another aspect of the invention is an electrosurgical instrument that has an instrument data memory containing data sets with structured data, of which at least some represent signaling instructions for acoustic and/or optical signals to be emmitet by an electrosurgical generator during operation.

The structured data can generally contain the references to operating specifications stored in the generator data memory. Such an electrosurgical instrument can therefore contain the necessary information that allows for an operating mode that is adapted to the respective electrosurgical instrument, without said operating mode having to be completely defined by the electrosurgical instrument.

Such an electrosurgical system, such an electrosurgical generator and such an electrosurgical instrument allow—each individually and in combination with each other—for the option that notification and error statuses as well as the behavior of those notifications do not have to be already known at the time of the development of the electrosurgical generator. In addition, subsequent adaptations of the notification and error statuses as well as of the behavior of these notifications can be easily made later on via a software update of the electrosurgical instrument. Even a “live” optimization of the notification and error statuses as well as of the behavior of these notifications during usability tests is possible.

The invention will now be explained in more detail based on exemplary embodiments referencing the figures. The figures show the following:

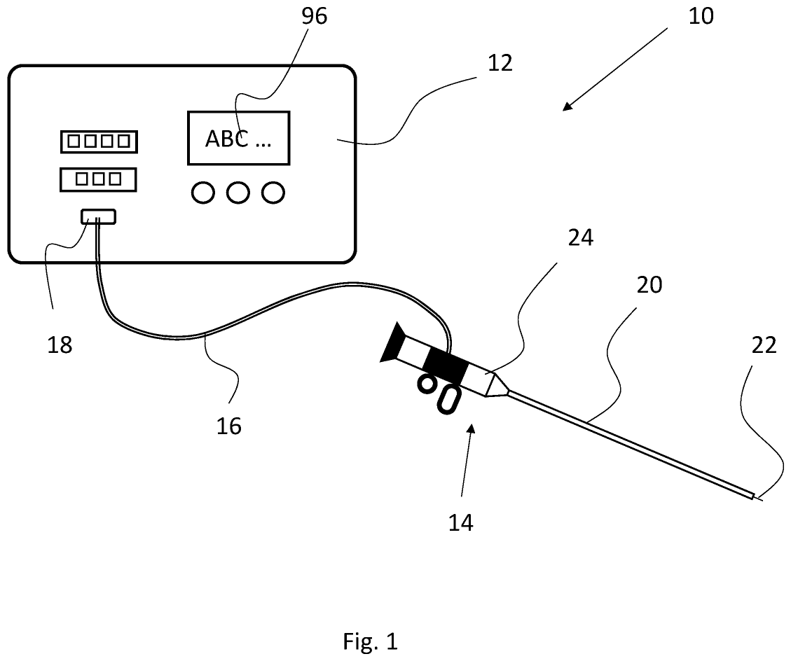

FIG. 1: an electrosurgical system with an electrosurgical generator and an electrosurgical instrument connected thereto;

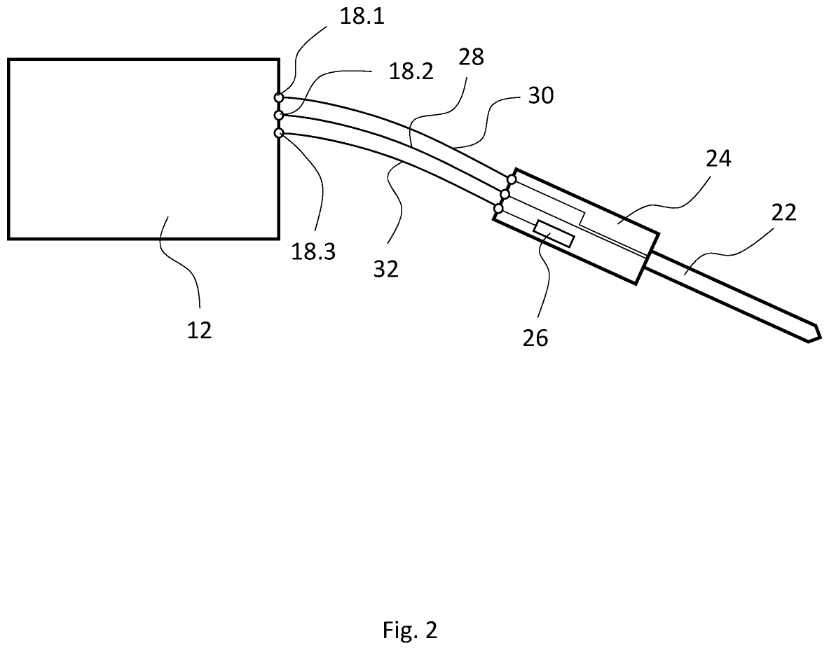

FIG. 2: an electrosurgical instrument;

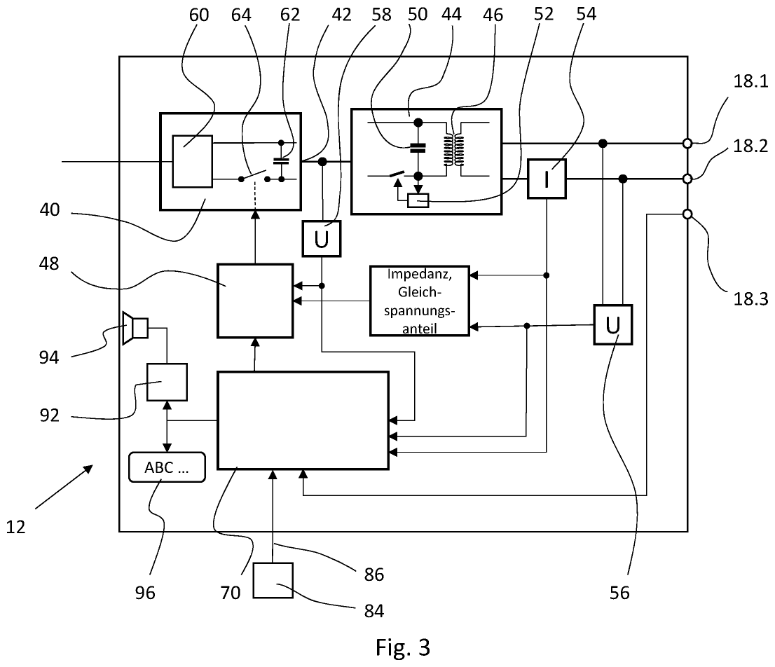

FIG. 3: a schematic diagram of an electrosurgical generator;

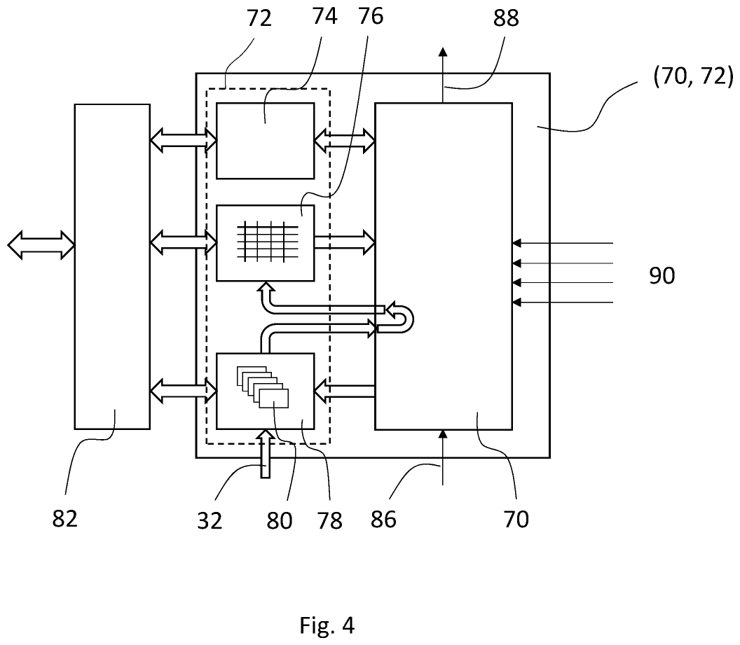

FIG. 4: a schematic illustration of a processor in combination with a generator data memory of the electrosurgical generator of FIG. 3;

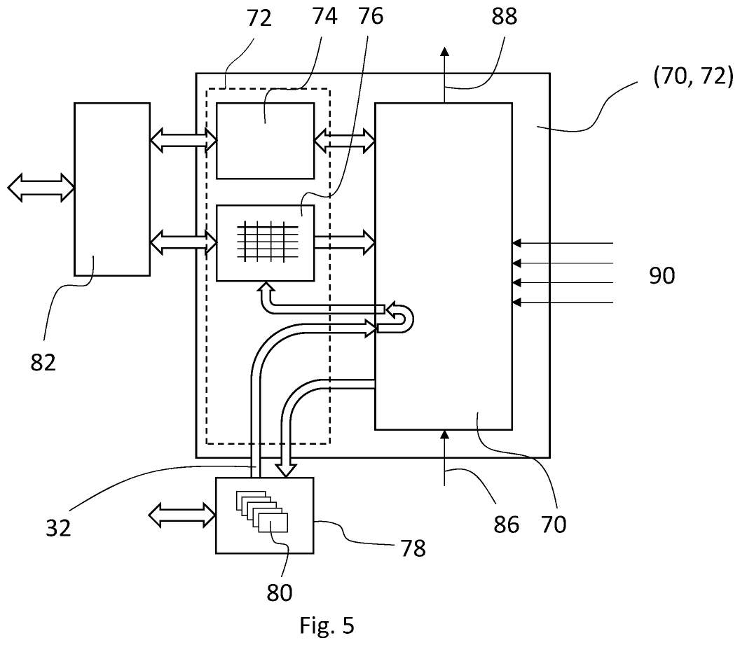

FIG. 5: a schematic illustration of an alternative configuration of a processor in combination with a generator data memory of the electrosurgical generator of FIG. 3.

FIG. 1 shows an electrosurgical system 10. The electrosurgical system 10 comprises an electrosurgical generator 12 and an electrosurgical instrument 14. Via a connection cable 16, the electrosurgical instrument 14 is connected to electrical out- and inputs 18 of the electrosurgical generator 12.

The electrosurgical instrument 14 has a shaft 20, at the end of which is an active electrode 22. The shaft 20 is attached to a handle 24 of the electrosurgical instrument 14.

The electrosurgical instrument 14 has an instrument data memory 26 that contains data sets 80 with structured data representing parameterized references to operating specifications, that are stored in a generator data memory 74 of the electrosurgical generator 12. In addition, the structured data represents signaling instructions that define which signals and/or information are to be output by the electrosurgical generator 12 at which the electrosurgical instrument 14 is operated, and how and under what conditions these signals and/or information are to be output. The instrument data memory 26 is non-volatile and may for example be a ROM (Read Only Memory), such as an EPROM (electrically programmable read-only memory), in particular an EEPROM (electrically erasable programmable read-only memory). An EEPROM is a non-volatile data memory that can be erased and newly written. The instrument data memory 26 is for example located in the handle 24 of the electrosurgical instrument 14.

The connection cable 16 contains both supply lines 28 and 30 and at least one data line 32. The supply lines 28 connect the active electrode 22 and another neutral electrode, that is not described in more detail, to the electrical outputs 18.1 and 18.2 of the electrosurgical generator 12. Via the data line 32, the instrument data memory 26 is connected to a corresponding data connection 18.3 of the electrosurgical generator 12. This is shown schematically in FIG. 2.

The data line 32 in the connection cable 16 as well as the data connection 18.3 may be a multi-core and/or multi-pole line/connection. In addition to or instead of the data line 32, a wireless interface may be provided for the data transfer from the electrosurgical instrument 14 to the electrosurgical generator 12. Such a wireless interface may for example be a Bluetooth interface, an NFC interface or an RFID interface.

During operation, the AC output voltage that is to be supplied to the active electrode 22 and a return electrode of the electrosurgical instrument 14 for the operation of the electrosurgical instrument 14 is provided by the electrosurgical generator 12. As shown in FIG. 3, the electrosurgical generator 12 has a high-voltage power supply 40 for this purpose, which can be connected to the usual public power grid and provides a high-frequency direct current with DC output voltage at its output 42. This direct output current is supplied to a high-frequency part 44 of the electrosurgical generator 12. The high-frequency part 44 of the electrosurgical generator 12 serves as an inverter and produces a high-frequency AC output voltage that is supplied to the outputs 18.1 and 18.2 of the electrosurgical generator 12 via an output transformer 46 of the high-frequency part 44. The electrosurgical instrument 14 can be connected to the outputs 18.1 and 18.2 of the electrosurgical generator 12, as shown in FIGS. 1 and 2.

To control the AC output voltage of the electrosurgical generator 12, a generator control unit 48 is provided that controls the AC output voltage at the outputs 18.1 and 18.2 of the electrosurgical generator 12 based on a maximum AC output voltage value such that, for example, a preset maximum output voltage value is not exceeded during operation.

The AC output voltage of the electrosurgical generator 12—and therefore also the alternating output current and the output power—can be controlled through the DC output voltage generated by the high-voltage power supply.

This is the purpose of the generator control unit 48 that controls the DC output voltage generated by the high-voltage power supply 40 in such a way that the resulting AC output voltage or an alternating output current are the voltage and/or current required by the respective operating mode of the electrosurgical generator at a respective point in time.

Each operating mode defines maximum values for the RMS of the AC output voltage through the outputs 18.1 and 18.2, the peak output voltage through the outputs 18.1 and 18.2, the alternating output current at the outputs 18.1 or 18.2, the DC voltage portion of the AC output voltage through the outputs 18.1 and 18.2 as well as the DC output voltage of the high-voltage power supply 40. The maximum values defined by a respective operating mode may be situation-dependent and change during use.

The generator control unit 48 controls the high-voltage power supply 40 in dependence on maximum values defined by a respective operating mode and on values of the AC output voltage, the peak output voltage, the alternating output current, the DC voltage portion of the AC output voltage or the DC output voltage detected during operation by detection units 54, 56 and 58, or on a combination of values of these parameters.

The specific maximum values and the time sequence for the generation of the DC output voltage of the electrosurgical generator 12 and their dependence on detected momentary values depend on the respective operating mode in which the electrosurgical generator 12 is currently being operated.

An operating mode is for example called up through the actuation of a corresponding switch by a user, for example a foot-operated switch 84.

In a respective operating mode, the operation of the electrosurgical generator 12 is controlled by a processor 70 in combination with an operating program stored in the generator data memory. The processor 70 can for example set the maximum values for the different operating parameters—such as the AC output voltage, the alternating output current, the output power, but also the DC voltage portion of the AC output voltage—wherein the respective current value of these parameters is detected during the operation of the electrosurgical generator 12.

The processor 70 is connected to the generator data memory 74, in which the operating program of the electrosurgical generator 12 is stored.

In order to control the output of the AC output voltage to be supplied to the electrosurgical instrument during operation, the processor 70 is, on the one hand, connected to the generator control unit 48. However, on the other hand, the processor 70 is also connected to the signaling and display means, namely in the specific case to a sound generator 92 including a loudspeaker 94 and to a display 96 for displaying text and/or symbols. The sound generator 92 can be configured to generate by means of a frequency generator or to play back acoustic signals specified in the form of audio files via the loudspeaker 94. The sound generator can also be configured to convert information stored in audio files into corresponding audio signals.

During the execution of the operating program 74 stored in the generator data memory 72, the processor 70 accesses at locations stored in the operating program 74 operating specifications 76, such as data representing values for operating parameters and/or control commands, which are also stored in the generator data memory 74 for a respective operating mode. The operating specifications 76 stored in the generator data memory 72 specify for example specific values for the DC output voltage of the high-voltage power supply or the AC output voltage, the alternating output current of the high-frequency part or similar data. However, the operating specifications 76 stored in the generator data memory 72 also include specific control commands, such as “if” or “while”, or “true” or “false”. Thus, the operating specifications 76 that represent data and control commands and are stored in the generator data memory 72 can, for example, be used to define control instructions such as “compare the current value of the AC output voltage to the amount 200 and return “true” if the current value of the voltage is smaller or equal to 200 and “false” if the current value is greater than 200”. Other data and control commands can, for example, be combined into the control instruction stating that the maximum DC output voltage of the high-voltage power supply shall be 100 Volt.

However, in order to generate and execute such control instructions, the processor 70 does not access the operating specifications 76 in the generator data memory 72 directly, but calls up a data structure 78 at the respective points of the operating program 74; references referring to corresponding operating specifications 76 in the generator data memory 72 are stored for the respective operating mode in said data structure 78; see e.g. FIG. 4. The references may, for example, be numbers, in particular 1 -byte binary numbers that can also be represented as hexadecimal numbers, since the latter require little memory space. In this case, the operating specifications 76 can be structured in the form of a table, in which each reference (i.e. for example each hexadecimal number) is assigned the corresponding control command or data.

A plurality of data sets 80 that each contain one reference or several references which, due to the structure of the respective data set—in particular the order in which the references are stored—can be assigned line numbers, are stored in the data structure 78, so that different operating modes can be implemented. The references assigned to a line number refer to the corresponding operating specifications 76 in the generator data memory 72 and cause the processor 70 to read the corresponding operating specifications 76 from the generator data memory 72, after the processor has accessed the vector address stored in the data structure 78 and designated by the associated line number. The operating specifications stored in the generator data memory 72 may, for example, be control instructions, control commands or parameter values, which the operating program is to apply at the respective point of the operating program, where the operating program 74 contains a reference to a line number in the data structure 78.

References to the operating specifications 76 stored in the generator data memory 72 are stored in the data structure 78 in an ordered sequence. The structure of a respective data set in the data structure 78 makes it possible to assign line numbers like addresses within the data structure to the references, so that for example jumps or returns to references in the data structure 78 are possible and not only a strictly sequential processing of the references by the operating program. The line numbers can serve as vector addresses within the structured data in the data structure 78, which the processor 70 accesses under the control of the operating program. The references assigned to the line numbers refer to specific operating specifications among the plurality of operating specifications 76 stored in the generator data memory 72. These operating specifications can be control specifications, namely for example individual control commands, combined control instructions or parameter values. The operating specifications 76 called up by the parameterized references in the data structure 78 control the operation of the electrosurgical generator 12 in the respective operating mode in combination with the operating program 74. The operating specifications may also be control commands that cause other references in the data structure 78 to be called up. However, this is only possible within the data—i.e. within the references—that belongs to a respective operating mode.

The parameterized references are preferably represented by hexadecimal numbers that, together, form structured data of a data set 80. A data set belongs to an operating mode and can for example contain the following:

| | | |
|---|---|---|
| 006F | 11 | |
| 0070 | 36 | 02 30 00 C8 00 |
| 0076 | 12 | |
| 0077 | 0F | 04 64 00 |
| 0078 | 56 | |
| 007C | 4B | 03 02 |
| 007F | 13 | |

The numbers shown in italics are line numbers that were generated while the data set 80 was being read and are not stored in the data memory 26 of the electrosurgical instrument 14; instead, they are generated by the operating program itself in accordance with the order of the references in a corresponding data set. Thus, the line numbers are the result of the order of the references (i.e. their structure) in a respective data set. The line numbers represented in the example by the numbers shown in italics are simply increasing numbers in accordance with the length of the structured data. The numbers shown in bold serve as references (or pointers), each of which refers to a specific operating specification 76 in the generator data memory 72, namely—in the illustrated example—to control commands. Thus, “11”, for example, refers to the control command “IF”, “36” refers to a comparison that is specified by the following assigned numbers “02 30 00 C 8 00 ”, “12” refers to the control command “THEN”, “0F” refers to a control command for setting a parameter value specified by the following assigned numbers “04 64 00”, and “56” refers to the control command “ELSE”.

The respective operating specification 76 called up by means of the parameterized reference also shows which additional information (such as “02 30 00 C8 00” in the example above) is also relevant.

The operating program 74 can read and translate the example described above as follows:
11—if→no additional information required
36—comparison→characters of additional information required (02=1 character type of comparison, 30 00=2 characters number 1, C8 00=2 characters number 2)
12—then→no additional information required
0F—set initial value→3 characters additional information (04=1 character which initial value; 64 00=2 characters set value)
. . .

Translated, this can mean “Compare whether number 1 (30 00) is greater than number 2 (C8 00). If the result is TRUE, set voltage to 100 V (64 00), else . . . ”

The binary numbers shown in the example as hexadecimal numbers (and herein referred to in short as “hexadecimal numbers”) in a respective data set 80 represent therefore first of all parameterized references, based on which the operating program 74 can access specific operating specifications in the generator data memory 72. The hexadecimal numbers stored in a data set in a structured (ordered) manner represent the parameterized references to operating instructions stored in the generator data memory 72, such as control commands and parameters, which, due to the structure of the data set, can be assigned line numbers that, as such, do not need to be explicitly stored in a data set 80, but that can be generated by the operating program 74 during the import of a respective data set 80.

The binary numbers stored in a respective data set 80 and shown as hexadecimal numbers in the example serve as pointers, each of which refers to a specific operating specification 76 in the generator data memory 72 of the electrosurgical generator 12 so that the corresponding hexadecimal number is linked to a corresponding operating specification 76 in the generator data memory 72 of the electrosurgical generator 12. Thus, these hexadecimal numbers are used to designate a corresponding memory entry in the generator data memory 76 of the electrosurgical generator 12 which represents an operating specification 76. These hexadecimal numbers are therefore a kind of pointer for guiding the operating program to the operating specifications 76 in the generator data memory 72 of the electrosurgical generator 12, where the operating program can, during its execution, in each case call up an operating specification for the operating program 74. The call-up of the references is controlled by the operating program and has the result that the processor 70 calls up the memory entries in the generator data memory 72 which the references refer to. Jumps within the references that are identified by their sequence or line numbers are also possible. Via the memory entries in the generator data memory 72 corresponding to it, the structured data in a data set 80 can thus be translated into operating specifications for the operating program.

In the data set shown by way of example above, the entries in the first column (“006F, 0070, 0076 . . . ”) are line numbers. The line numbers are not stored in the instrument data memory 26 of the electrosurgical instrument 14, but are generated by the operating program 74 itself, since the line numbers are simply increasing numbers in accordance with the length of the structured data:
0070 36 02 30 00 C8 00 (data length=6, i.e. the next line number is 0076)
0076 12 (data length=1, i.e. the next line number is 0077)

The entries in each line (“11, 36 02 30 00 C8 00, 12 . . . ”) refer to operating specifications 76 in the generator data memory 72. The entry in the generator data memory labeled “11” may for example be an “IF” instruction, while the entry labeled “12” may be a “THEN” instruction. The “IF” instruction and the “THEN” instruction are each one operating specification. Based on the associated memory entries in the generator data memory 72, the hexadecimal numbers “36 02 30 00 C8 00” that, in the structured data of the illustrated data set, are located between 11 and 12 can be translated into a control instruction, such as “Compare the last read value of the voltage (30 00) with the number 200 (C8 00) and return “TRUE” if the voltage is smaller or equal to 200, else return “FALSE”. When the operating program 74 calls up the memory entries for the string “0F 04 64 00” from the generator data memory 72 at the address “0077” (generated by the operating program), this could denote a setting for the electrosurgical generator 12; the setting may e.g. be that a maximum value for the DC output voltage (0F 04) is set to 100V (64 00).

An important aspect is the output of notifications to a user via the loudspeaker 94 or the display 96. The described structure of the electrosurgical generator 12 is also helpful in this respect, since data representing signaling instructions can be stored in the instrument data memory 26 of the electrosurgical instrument 14, and thus do not need to be stored in the electrosurgical generator 12.

The storage of data representing signaling instructions in the instrument data memory 26 of the electrosurgical instrument 14 according to the invention makes it, in particular, possible to flexibly define activation sounds and other acoustic signals via the electrosurgical instrument 14. In practical terms, a byte sequence, for example, is stored in the structured data 80 in the instrument data memory—i.e. for example in the EEPROM of the electrosurgical instrument 14. The byte sequence defines for example the activation sound or other acoustic signals. The following options, among other options, can be implemented:
1. Playback of an audio filed defined in the electrosurgical generator 12 (e.g. ActivationCut.wav)
2. Playback of an audio file defined in the electrosurgical generator 12 with a defined pulse-pause ratio (e.g. ActivationCut.wav with a period duration of 500 ms and a pause duration of 100 ms)
3. Playback of a defined frequency via the frequency generator implemented in the electrosurgical generator (e.g. 500 Hz)
4. Playback of a defined frequency via the frequency generator 92 implemented in the electrosurgical generator with a defined pulse-pause ratio (e.g. 500 Hz with a perk od duration of 500 ms and a pause duration of 100 ms)
5. Playback of a frequency mixture via the frequency generator 92 implemented in the electrosurgical generator 12 (e.g. 500 Hz together with 100 Hz and 2000 Hz)
6. Playback of a frequency mixture via the frequency generator 92 implemented in the electrosurgical generator 12 with a defined pulse-pause ratio (e.g. 500 Hz together with 1000 Hz and 2000 Hz with a period duration of 500 ms and a pause duration of 100 ms)
7. Change of the activation sound in dependence on the operating mode (e.g. an activation sound A under normal conditions and activation sound B if a certain resistance value is reached).

This leads to a very high degree of flexibility so that even very complex sound sequences can be stored on the electrosurgical instrument 14 with the aid of the programming interface 82 and the data sets 80 in the data structure 78.

With regard to the output of acoustic signals to a user, the data structure 78—or, more precisely: a data set in the data structure—may contain the following entries for the implementation of the aforementioned examples:

1) Playback of an audio file stored in the generator data memory 72 of the electrosurgical generator 12

| Set Activation Sound File (Start. 3, 0 ms, 0 ms) | | |
|---|---|---|
| 0048 | 46 | 00 03 00 00 00 00 00 |

2) Playback of an audio file stored in the generator data memory 72 of the electrosurgical generator 12 with a defined pulse-pause ratio

| Set Activation Sound File(Start, 3, 500 ms, 100 ms) | | |
|---|---|---|
| 0048 | 46 | 00 03 00 F4 01 64 00 |

3) Playback of a defined frequency via the frequency generator 92 implemented in the electrosurgical generator 12

| Set Activation Sound Frequency(Play, 500 Hz, 0 ms, 0 ms) | | |
|---|---|---|
| 0048 | 47 | 00 F4 01 00 00 00 00 |

4) Playback of a defined frequency via the frequency generator 92 implemented in the electrosurgical generator 12 with a defined pulse-pause ratio

Set Activation Sound Frequency(Play, 500 Hz, 500 ms, 100 ms)

| | | |
|---|---|---|
| 0048 | 47 | 00 F4 01 F4 01 64 00 |

5) Playback of a frequency mixture via the frequency generator 92 implemented in the electrosurgical generator 12

Buffer(Initialize, activationTones_000, 3)
Buffer(Set Value Directly, activationTones_000. 0, 500)
Buffer(Set Value Directly, activationTones_000, 1, 1000)
Buffer(Set Value Directly, activationTones_000, 2, 2000)
Set Activation Sound Multi Frequency(Play, activationTones_000, 0 ms, 0 ms)

| | | |
|---|---|---|
| 0048 | 58 | 08 00 03 OD |
| 004D | 58 | 30 00 GO 00 F4 01 |
| 0054 | 58 | 30 00 01 00 E8 03 |
| 005B | 58 | 30 00 02 00 DO 07 |
| 0062 | 57 | 03 00 00 00 00 OD |

6) Playback of a frequency mixture via the frequency generator 92 implemented in the electrosurgical generator 12 with a defined pulse-pause ratio

Buffer(Initialize, activationTones_000, 3)
Buffer(Set Value Directly. activationTones_000, 0, 500)
Buffer(Set Value Directly. activationTones_000, 1, 1000)
Buffer(Set Value Directly. activationTones_000, 2, 2000)
Set Activation Sound Mufti Frequency(Play, activationTones_000, 500 ms, 100 ms)

| | | |
|---|---|---|
| 0048 | 58 | 03 00 03 00 |
| 004D | 58 | 30 00 00 00 F4 01 |
| 0054 | 58 | 30 00 01 00 E8 03 |
| 0058 | 58 | 30 00 02 00 DO 07 |
| 0062 | 57 | 03 00 F4 01 64 00 |

7) Playback of an activation sound in dependence on the operating mode

If( ) ... Then( )

[conditions]

Compare(<=. load Resistance_000, 500)

[then]

Set Activation Sound Frequency(Play, 400 Hz, 0 ms, 0 ms)

[else]

Set Activation Sound Frequency(Play, 500 Hz, 100 ms, 51ms)

| | | |
|---|---|---|
| 004E | 11 | |
| 004F | 36 | 02 30 00 F4 01 |
| 0055 | 12 | |
| 0056 | 47 | 00 90 01 00 00 00 00 |
| 005E | 56 | |
| 005F | 47 | 00 F4 01 64 00 32 00 |
| 0067 | 13 | |

With regard to the display of texts in a display format that varies depending on the display option, a notification text may be represented by a characters string that, in addition, contains special characters or non-printable characters (control characters) that specify in particular the shortening of a notification text by the processor 70 under the control of the operating program 74, the operating specifications 76 and the structured data, if this is required by a display 96.

This makes it possible for an electrosurgical instrument 14 to be operated at different electrosurgical generators 12, which have different displays. Thus, suitable displays can show a longer notification text than smaller displays.

The character string makes the notification text to be displayed flexible, since the text to be displayed is stored on the electrosurgical instrument 14 along with at least one truncating instruction. Truncating instructions may for example be special characters or non-printable characters (herein also referred to as control characters) inserted in the character string, which the processor 70 can use in accordance with a predefined set of rules to perform the necessary shortening of the text to be displayed. The character string can be truncated at the end or at the beginning as well as in the middle. Combined truncations are possible and are controlled by priority.

Thus, in addition to the text to be displayed (the actual string), a character string may contain control characters that define an adaptation of the text to be displayed if it is to be displayed on a respective display 96 of the electrosurgical generator 12.

This shall be explained in the following, based on a number of simplified examples.

Simplified example 1 (string truncated at the end):
Conventional operating mode name: “BiSoftCoag”
Operating mode name with truncating instruction: “Bi#Soft#Coag”
If sufficient space was available, “BiSoftCoag” would be displayed on the display of the electrosurgical generator (12)
If less space was available, “BiSoft” would be displayed on the display of the electrosurgical generator (12)
If very little space was available, “Bi” would be displayed on the display of the electrosurgical generator (12)

Thus, the special character “#” is never displayed and the character string to be displayed can be truncated at the places where the special character “#” is located.

Simplified example 2 (string truncated in other places):
Conventional operating mode name: “PK SoftCoag”
Operating mode name with truncating instruction: “{PK}SoftCoag”
If sufficient space was available, “PK SoftCoag” would be displayed on the display of the electrosurgical generator (12)
If less space was available, “SoftCoag” would be displayed on the display of the electrosurgical generator (12)

Thus, the character string in the curly brackets is, where necessary, not displayed.

Simplified example 3 (combined truncation):
Conventional operating mode name: “PK SoftCoag”
Operating mode name with truncating instruction: “{PK}Soft#Coag”
If sufficient space was available, “PK SoftCoag” would be displayed on the display of the electrosurgical generator (12)
If less space was available, “PK Soft” would be displayed on the display of the electrosurgical generator (12)

If very little space was available, “Soft” would be displayed on the display of the electrosurgical generator (12)

Example 3 shows how the measures for truncating the character string to be displayed explained in examples 1 and 2 can be combined.

With the aid of the signaling and display means in the form of the loudspeaker 94 including the sound generator 92 and the display 96, a user can be notified of operating states, errors etc. The control instructions that define such notifications are represented by data that is stored in the data memory 26 of the electrosurgical instrument 14. This means that it is not necessary for notification and error states as well as for the behavior of these notifications to be already known at the time of the development of the electrosurgical generator. In addition, subsequent adaptations of the notification and error statuses as well as of the behavior of these notifications can be easily made later on via a software update of the electrosurgical instrument. Even a “live” optimization of the notification and error statuses as well as of the behavior of these notifications during usability tests is possible.

Furthermore, the storage of data representing signaling instructions in the data memory 26 of the electrosurgical instrument 14 according to the invention makes it possible to define notifications and error messages as well as the behavior of these notifications flexibly on the electrosurgical instrument 14. In practical terms, a byte sequence, for example, is stored in the structured data 80 in the instrument data memory 26 of the electrosurgical instrument 14—i.e. for example in the EEPROM. The byte sequence defines for example the conditions and characteristics for a specific notification or error message.

The notification and error message types explained by way of example below can be defined on the electrosurgical instrument.

1. Display of an info text
flexible trigger condition
flexible sound configuration (silent, standard info sound, customized sound)
flexible high-frequency/ultrasound handling (with deactivation of HF/US, without deactivation of high-frequency/ultrasound)

2. Display of a low-priority error with standard low-priority sound
flexible trigger condition
flexible high-frequency/ultrasound handling (with deactivation of high-frequency/ultrasound, without deactivation of high-frequency or ultrasound)

3. Display of a medium-priority error with standard medium-priority sound
flexible trigger condition
flexible high-frequency handling (with deactivation of high-frequency/ultrasound, without deactivation of high-frequency/ultrasound)

4. Display of a high-priority error with standard high-priority sound

Among other things, the following trigger conditions are possible:
1. During the connection of an electrosurgical instrument 14 to the electrosurgical generator 12
2. In the “idle” state of the electrosurgical generator 12 (e.g. after a defined period of time has elapsed since the connection of an electrosurgical instrument 14)
3. Shortly before the output of high-frequency/ultrasound (e.g. prevention of the activation if the electrosurgical instrument 14 has expired)
4. Shortly after the output of high-frequency/ultrasound (e.g. notification that the application has not yet been completed)

5. During the activation in dependence on the operating mode, e.g.
in dependence on the tissue impedance
In dependence on the activation duration
In case of the occurrence of certain events (e.g. sparks)

It is also possible that the texts displayed are stored in multiple languages on the electrsurgical instrument—i.e. in the instrument data memory 26.

This is illustrated by the following exemplary implementations:

A) Examples of different notification types

| Generate Error(UInt 16. 666) | | |
|---|---|---|
| 0068 | 4A | 00 9A 02 |

Silent info text with high-frequency/ultrasound deactivation

| Generate Error(UInt 16. 666) | | |
|---|---|---|
| Stop Activation^{°} | | |
| 0068 | 4A | 00 9A 02 |
| 006C | 53 | |

Silent info text with high-frequency/ultrasound deactivation and customized sound configuration

Generate Error(UInt 16. 666)
Set Activation Sound Frequency(Play, 750 Hz. 100 ms, 50 ms)
Busy Wait(Time, 1000)
Stop Activation^{°}

| | | |
|---|---|---|
| 0068 | 4A | 00 9A 02 |
| 006C | 47 | 00 EE 02 64 00 32 00 |
| 0074 | 48 | 00 E8 03 |
| 0078 | 53 | |

Low-priority error without high-frequency/ultrasound deactivation

| Generate Error(UInt 16. 676) | | |
|---|---|---|
| 0068 | 4A | 00 A4 02 |

Low-priority error with high-frequency/ultrasound deactivation

| Generate Error(UInt16, 676) | | |
|---|---|---|
| Stop Activation° | | |
| 0068 | 4A | 00 A4 02 |
| 006C | 53 | |

Medium-priority error without high-frequency/ultrasound deactivation

| Generate Error(UInt 16, 188) | | |
|---|---|---|
| 0068 | 4A | 00 BC 00 |

Medium-priority error with high-frequency/ultrasound deactivation

| Generate Error(UInt16, 188) | | |
|---|---|---|
| Stop Activation° | | |
| 0068 | 4A | 00 BC 00 |
| 006C | 53 | |

High-priority error

| Generate Error(UInt16. 14) | | |
|---|---|---|
| 0068 | 4A | 00 OE 00 |

B) Examples of trigger conditions

During the connection of an electrosurgical instrument

errorOnConnecti_000

[options]

Additional Execution: On Instrument Connection

[parameters]
[content]

Generate Error(UInt16, 1)

| | | |
|---|---|---|
| 001D | 00 | 0B 00 |
| 0020 | 01 | 00 |
| 0022 | 04 | |
| 0023 | 00 | 00 |
| 0025 | 04 | 00 |
| 0027 | 4A | 00 01 00 |

In the “idle” state of the electrosurgical generator (e.g. after a defined period of time has elapsed since the connection of an electrosurgical instrument)

errorIdle_000

[options]

Additional Execution: In Idle State

[parameters]
[content]

Busy Watt(Time. 5000)
Generate Error(UInt16. 1)

| | | |
|---|---|---|
| 001D | 00 | 0F 00 |
| 0020 | 01 | 00 |
| 0022 | 01 | |
| 0023 | 00 | 00 |
| 0025 | 08 | 00 |
| 0027 | 48 | 00 88 13 |

Shortly before the output of high-frequency or ultrasound

errorBeforeActvation_000

[options]

Additional Execution: Before Activation

[parameters]
[content]

.Generate Error(UInt16. 1)

| | | |
|---|---|---|
| 0010 | 00 | 0B 00 |
| 0020 | 01 | 00 |
| 0022 | 02 | |
| 0023 | 00 | 00 |
| 0025 | 04 | 00 |
| 0027 | 4A | 00 01 00 |

Shortly after the output of high-frequency or ultrasound

errorAfterActivation_000

[options]

Additional Execution: Alter Activation

[parameters]

Generate Error(UInt 16, 1)

| | | |
|---|---|---|
| 001D | 00 | 0B 00 |
| 0020 | 01 | 00 |
| 0022 | 03 | |
| 0023 | 00 | 00 |
| 0025 | 04 | 00 |
| 0027 | 4A | 00 01 00 |

During the activation in dependence on the tissue impedance

If( ) ... Then( )

[conditions]

Compare(>=, loadResistance_00:, 2000)

[then]

Generate Error(1.11nt16, 1)

| | | |
|---|---|---|
| 007C | 11 | |
| 0070 | 02 | 10 00 DO 07 |
| 0083 | 12 | |
| 0084 | 4A | 00 01 00 |
| 0088 | 13 | |

Thus, the operation of the electrosurgical generator 12 in a respective operating mode depends first of all on the operating program 74 stored in the generator data memory 72. However, in addition, the operating behavior of the electrosurgical generator 12 in a respective operating mode also depends on the data set 80 in the data structure 78 called up for a respective operating mode as well as on the operating instructions 76 that are also stored in the generator data memory 72 and, in particular, also on the data representing signaling instructions that is stored in the instrument data memory 26 of the electrosurgical instrument 14. In this context, the operating behavior includes both the supply of AC output voltage to the electrosurgical instrument 14 and the display of text via the display 96 or the output of acoustic signals via the loudspeaker 94.

The advantage of such an electrosurgical generator 12 is that a user can easily define new operating modes including the associated notifications by generating new data sets 80 in the data structure 78 and that a single operating mode can, for example, be changed solely via a change of the corresponding data set 80 in the data structure 78, without the operating program 74 in the generator data memory 72 or the operating specifications 72 in the generator data memory 72 having to be changed. It is, in particular, possible to easily define signaling instructions for an electrosurgical instrument 14 by means of data in the data memory 26 of the electrosurgical instrument 14.

This means that it is not necessary for notification and error states as well as for the behavior of these notifications to be already known at the time of the development of the electrosurgical generator. In addition, subsequent adaptations of the notification and error statuses as well as of the behavior of these notifications can be easily made later on via a software update of the electrosurgical instrument. Even a “live” optimization of the notification and error statuses as well as of the behavior of these notifications during usability tests is possible.

On the other hand, global parameters, such as any potential control instructions that might be available or operating parameters depending on the electrosurgical generator, such as its maximum AC output voltage or a minimum permissible DC output voltage, can be stored as operating specifications 76 in the generator data memory 76, where they can, if need be, also be changed centrally for all possible operating modes at once.

Another advantage of the design of the electrosurgical generator 12 is that a data set 80, the structured data of which indirectly defines an operating mode suitable for the electrosurgical instrument 14, can also be stored in an electrosurgical instrument 14; see FIG. 5. Specifically, the instrument data memory 26 of the electrosurgical instrument 14 can contain a data set with structured data that is compatible with the data structure 78 in the generator data memory 72 and, just as other structured data in the data structure 78, indirectly defines a respective operating mode by means of corresponding references to the operating specifications 76 in the generator data memory 72. This is a great advantage especially with regard to acoustic and optical signals for notifying a user about operating states or errors etc., since control instructions for an individual electrosurgical instrument 14 that define such signals can be represented by data that can be stored in the instrument data memory 26 of this electrosurgical instrument 14 in a manner appropriate for the respective individual electrosurgical instrument 14.

Therefore, the electrosurgical generator 12 is configured such that while an electrosurgical instrument 14 is being connected, the electrosurgical generator 12 will, in each case, first of all read the instrument data memory 26 of the electrosurgical instrument 14—if available—and enter the structured data from the data set stored in the instrument data memory 26 into the data structure 78 in the generator data memory 72. Thus, an operating mode precisely tailored to the respective electrosurgical instrument 14 will be available to the electrosurgical generator 12 during operation.

In order to allow access to the content of the instrument data memory 26, at least one data line 22 with a corresponding connection 18.3 is provided. As an alternative or in addition, a wireless interface such as a Bluetooth interface or an NFC interface may be provided for the access to the content of the instrument data memory 26.

When the structured data is transferred from the instrument data memory 26 of the electrosurgical instrument 14 into a corresponding data set in the data structure 78 of the electrosurgical generator 12, the line numbers can, if applicable, be generated to match the operating program 74 of the electrosurgical generator 12.

It is a great advantage that the data set stored in the instrument data memory 26 only contains references ordered in a structured manner (pointers to further memory entries in the generator data memory 72), but does not directly contain any control instructions or operating parameters for a respective operating mode of any kind, since the control instructions and the operating parameters are stored centrally in the generator data memory 76 of the electrosurgical generator 12.

Alternatively, the electrosurgical generator 12 can also be configured such that it directly reads out the instrument data memory 26 of the electrosurgical instrument 14 during operation—i.e. during the execution of the operating program. This is the case in the example shown in FIG. 5. In this case, the structured data of the data set in the instrument data memory 26 does not need to be transferred into the data structure 78 of the electrosurgical generator 12 first. However, the line numbers to be generated must match the corresponding call-ups in the operating program and the entries in the generator data memory 72 in this case.

An advantage of the electrosurgical system 10 of the type described herein is that different parameters that define the operation of the electrosurgical generator 12 can be managed independently of one another. Thus, the operating program 74 stored in the generator data memory 72 is stored independently of the operating specifications 76 in the generator data memory 72. The operating specifications 76 are, in turn, stored independently of the structured data in the data structure 78.

Therefore, a programming interface 82 is preferably provided that preferably provides a graphical user interface and that is created via a data set with structured data implemented as a plurality of parameterized references and can be stored in the instrument data memory 26 of the electrosurgical instrument 14.

Preferably, the programming interface 82 is configured such that it assigns different rights to different users. Thus, different rights can be assigned for programming the operating program 74 that is stored in the generator data memory 72, for entering the operating specifications 76 that are also stored in the generator data memory 72 and for the structured data that is stored in the data structure 78. Thus, it can in particular be ensured that changes to the operating specifications 76 or changes to the operating program 74 can only be made by developers who are familiar with the respective electrosurgical generator 12. The operating program 74 and the operating specifications 76 can thus be programmed by developers who are familiar with the respective electrosurgical generator 12, while a developer who is familiar with the electrosurgical instrument 14 can define the operating modes for an electrosurgical instrument 14 by creating a corresponding data set with structured data. Preferably, data sets created by a developer who is familiar with the electrosurgical instrument 14 are stored in the instrument data memory 26 of the respective electrosurgical instrument 14, while further operating modes can also be stored directly in the data structure 78 on the electrosurgical generator 12. To this end, the electrosurgical generator 12 can for example have a USB programming interface. Either the data line 32 in the connection cable 16 with a corresponding interface, or—as an alternative or in addition—a wireless interface, such as a Bluetooth interface or an

NFC interface, is available for the structured data stored in the instrument data memory 26 of the respective electrosurgical instrument 14. Thus, the structured data from the data set in the instrument data memory 26 of the electrosurgical instrument 14 can be transferred into the data structure 78 on the electrosurgical generator 12 when the electrosurgical instrument 14 is connected.

This is, in particular, relevant with regard to different electrosurgical instruments 14, since the electrosurgical instruments 14 might be programmed by other developers than the electrosurgical generator 12. The developers of the electrosurgical generator 12 can store all the specific parameter data and control commands that are important for the electrosurgical generator 12—if need be in dependence on the operating program 74 stored in the generator data memory 72—as operating specifications 76 in the generator data memory 72. Such parameter values can for example be maximum or minimum permissible values for the DC output voltage, the AC output voltage etc.

Independently of this, developers of an electrosurgical instrument 14 can use the structured data in the data set in the instrument data memory 26 of the electrosurgical instrument to specify in detail how a specific operating mode can be executed for this electrosurgical instrument 14 within the framework of the threshold values defined by the operating program 74 in the generator data memory 72 and the operating specifications 76 in the generator data memory 72. The developers of the electrosurgical instrument 14 do not need to give any further consideration to the specifications provided by the operating program 74 and the operating specifications 76. Instead, the developers of the electrosurgical instrument 14 can accept these specifications provided for the respective electrosurgical generator 12.

A programming interface 82 via which a developer can fully define an operating mode for a respective electrosurgical instrument 14 is available to the developers of an electrosurgical instrument 14 for the definition of an operating mode for the respective electrosurgical instrument 14. This definition includes for example all the current and voltage parameter values as well as timing specifications and transition conditions for the operation of the electrosurgical instrument. Thus, the operating mode can be developed with the aid of an easy-to-use tool virtually without any software development knowledge by a developer for an electrosurgical instrument. The programming interface 82 provides to the developer a number of parameter sets that the developer can use to define different phases, for example the phase of the first cut, the cutting phase, the coagulation phase, but also short-circuit or power monitoring for the respective electrosurgical instrument. A development tool belonging to the programming interface 82 generates a memory space-saving set of structured data from the specifications, wherein said set of data forms a data set that can be stored in the data memory 26 of the electrosurgical instrument 14 in a non-volatile manner.

If a new operating mode for an electrosurgical instrument 14 defined by structured data in a data set 80 also requires a change of the operating program 74 in the generator data memory 72 or of the operating specifications 76 in the generator data memory 72, such changes can, for example, be made by a developer who is familiar with the electrosurgical generator 12 via a programming interface 82. Thus, it can be ensured that the structured data that defines an operating mode is compatible with the operating specifications 76 in the generator data memory 72 and the operating program 74 in the generator data memory 72. The developer of the electrosurgical instrument 14 can consult the developer familiar with the electrosurgical generator 12 in this regard. As a result, it is ensured that a developer who is not familiar with the electrosurgical generator 12—e.g. a developer for an electrosurgical instrument 14—cannot create erroneous operating specifications 76 or change the operating program 74 in a way that causes unintended effects or errors.

When an electrosurgical instrument 14 is connected to the electrosurgical generator 12, the electrosurgical generator 12 reads the instrument data memory 26 in the electrosurgical instrument 14 and calls up the operating specifications 76 in the generator data memory 72 that are designated by the references contained in the structured data, so that the current and voltage curves including any timing requirements and other conditions defined in those operating specifications 76 are applied. This allows for reduced development times and costs. For the most part, the electrosurgical instrument 14 can be integrated independently of an electrosurgical generator 12. In addition, this allows for a shorter time-to-market, since the operating modes can also be developed and finalized after the introduction of an electrosurgical generator 12. Optimizations of an operating mode and new operating modes can be easily introduced by means of updated or new electrosurgical instruments 14. An operating mode for an electrosurgical instrument can be defined with almost no software development knowledge.

If the electrosurgical system 10 is to be operated with an electrosurgical instrument 14 connected to the electrosurgical generator 12, the appropriate operating mode will already be available once the electrosurgical instrument 14 has been connected, since the associated data sets 80 with the structured data can be read by the instrument data memory 26 of the electrosurgical instrument 14. Therefore, a user will, for example, only have to actuate a switch 84, in order to operate the electrosurgical instrument 14 in the appropriate operating mode of the electrosurgical generator 12. After the connection of the electrosurgical instrument 14, the user does not need to set or program anything.

The switch 84 is connected to the processor 70 of the electrosurgical generator 12 via a line 86, so that the execution of the operating program 74 stored in the generator data memory 72 can be started and stopped through the actuation of the switch 84. The switch 84 may be a foot-operated switch, but may also be a hand-operated switch, that is, for example, located at the electrosurgical hand-held instrument 14. A wireless control connection can be provided instead of the line 86.

Another alternative to a switch 84 is an automatic start of the operating program 74, which a user can activate in advance. In this case, the electrosurgical instrument 14 first of all outputs a small measurement voltage in order to detect tissue contact (current flow) with the aid of said measurement voltage. If tissue contact—i.e. current flow—is detected, the actual operating program 74 for the electrosurgical instrument will be called up. If the tissue contact disappears, the actual operating program 74 for the electrosurgical instrument will be ended, and a small measurement voltage will once again be output so that a new tissue contact can be detected with the aid of said measurement voltage.

Under the control of the operating program, the processor 70 indirectly accesses individual operating specifications 76 in the generator data memory 72 during the use of an operating mode by first of accessing the references in the data structure 78 and subsequently calling up the operating specification 76 or operating specifications 76 referred to by the respective reference. With regard to the output of notifications to a user, the processor 70 indirectly accesses individual audio files in the generator data memory 72 during the use of an operating mode, by first of all accessing data in the instrument data memory 26 that represents signaling instructions and subsequently calling up the audio files and activating the sound generator specified by the signaling instructions.

In dependence on the operating program 74 as well as on the operating specifications 76 in the generator data memory and the references and signaling instructions in the data structure 78 as well as on signals 90 coming from the detection units 54, 56 and 58, the processor 70 generates control signals 88 for the generator control unit 46.

### REFERENCE NUMBERS

10 electrosurgical system

12 electrosurgical generator

14 electrosurgical instrument

16 connection cable

18.1, 18.2 electrical outputs

18.3 connection

20 shaft

20.1, 20.2 outputs

22 active electrode

24 handle

26 instrument data memory

28, 30 supply lines

32 data line

40 high-voltage power supply

42 output

44 high-frequency part

46 output transformer

48 generator control unit

50 capacitor

52 synchronizing circuit

54 output current detection unit

56 AC output voltage detection unit

58 DC output voltage detection unit

60 high-voltage rectifier circuit

62 output capacitor

64 switch

70 processor

72 generator data memory

74 operating program

76 operating specifications

78 data structure

80 data set

84 switch

86 line

88 control signals of the processor

90 signals of the detection units

92 sound generator including frequency generator

94 loudspeaker

96 display for text and/or symbols

1. A method of operating an electrosurgical system with an electrosurgical instrument, that has at least one instrument data memory, and with an electrosurgical generator, to which the electrosurgical instrument is connected during operation, wherein the electrosurgical generator has a processor and at least one generator data memory connected to the processor, wherein, in order to control the electrosurgical generator, the processor executes an operating program stored in the generator data memory;
wherein
a data set containing structured data is generated or provided, wherein the structured data is implemented as a plurality of parameterized references referring to operating specifications stored in the generator data memory of the electrosurgical generator, which comprise potentially applicable control commands and may comprise additional operating parameter values,
the data set with structured data is or will be stored in the instrument data memory of the electrosurgical instrument,
the data set with structured data stored in the instrument data memory of the electrosurgical instrument is read by the electrosurgical generator when the electrosurgical generator is started up or during its operation, and
the parameterized references stored in the structured data are called up under the control of the operating program for the execution of the control commands stored in the electrosurgical generator in accordance with the operating parameters that are also available in the structured data, so that the electrosurgical generator is controlled in dependence on the operating program, the operating specifications, stored in the generator data memory and the parameterized references to individual ones of the control commands stored in the generator data memory which are contained in the structured data.

2. A method according to claim 1, wherein after being read by the electrosurgical generator, the data set with structured data stored in the instrument data memory of the electrosurgical instrument is stored in the generator data memory of said electrosurgical generator.

3. A method according to claim 1, wherein structured data stored in the instrument data memory of the electrosurgical instrument is changed when it is being read by the electrosurgical generator or after it has been stored in the generator data memory of the electrosurgical generator.

4. A method according to claim 1, wherein the structured data comprises signaling instructions that are converted into configuration and/or operating status-dependent acoustic and/or visual notifications to a user during the operation of the electrosurgical generator.

5. A method according to claim 1, wherein the structured data comprises definitions for activation sounds.

6. A method according to claim 1, wherein the structured data comprises conditions and characteristics for the generation and/or display of notifications and/or error messages.

7. A method according to claim 1, wherein the structured data comprises control characters for data representing text notifications, wherein the control characters define the display of text notifications on various displays, and in that text notifications are generated during the operation of the electrosurgical generator in dependence on the control characters and a display for text present at the electrosurgical generator.

8. An electrosurgical system with an electrosurgical instrument, that has at least one instrument data memory, and with an electrosurgical generator, to which the electro surgical instrument is connected, wherein the electrosurgical generator has a processor and at least one generator data memory, wherein, in order to control the electrosurgical generator, the processor executes an operating program stored in the generator data memory; characterized wherein
a data set with structured data is stored in the instrument data memory of the electrosurgical instrument, wherein the structured data is implemented as a plurality of parameterized references referring to operating specifications stored in the generator data memory of the electrosurgical generator, which contain control commands and/or, if need be, comprise additional operating parameters,
and in that the electrosurgical generator is configured
to read the data set with structured data stored in the instrument data memory of the electrosurgical instrument when the electrosurgical system is started or during its operation, and
to call up the parameterized references stored in the structured data under the control of the operating program in order to execute the control commands stored in the electrosurgical generator taking into account operating parameters specified in the structured data.

9. An electrosurgical system according to claim 8, where the electrosurgical generator has a sound generator and a loudspeaker connected to the latter for the output of acoustic signals, wherein the sound generator is operatively connected to the processor and configured to generate acoustic signals and to output them via the loudspeaker under the control of the processor in dependence on data in the instrument data memory of the electrosurgical instrument which represents signaling instructions.

10. An electrosurgical system according to claim 9, where audio files that the sound generator can convert into acoustic signals under the control of the processor and the operating program in dependence on data in the instrument data memory of the electrosurgical instrument which represents signaling instructions are stored in the generator data memory.

11. An electrosurgical system according to claim 8, where the electrosurgical generator has a display for displaying text and/or symbols, which is connected to the processor and configured to output text notifications under the control of the processor and the operating program in dependence on data in the instrument data memory of the electrosurgical instrument which represents signaling instructions.

12. An electrosurgical system according to claim 8, with one programming interface or several programming interfaces, by means of which the content of the generator data memory and of the data structure can be programmed.

13. An electrosurgical instrument for an electrosurgical system according to claim 1, with an instrument data memory containing data sets with structured data, wherein the structural data include data representing signaling instructions for acoustic and/or optical signals to be emittet by an electrosurgical generator during operation.

14. A method of creating a structured data set for an electrosurgical instrument of an electrosurgical system according to claim 1, wherein a programming interface is provided and in that a data set with structured data that is implemented as a plurality of parameterized references is created via the programming interface and stored in the instrument data memory of the electrosurgical instrument.
